(19) United States
(12) Patent Application Publication
(10) Pub. No.: US 2024/0168028 A1
(43) Pub. Date: May 23, 2024

(54) METHOD FOR PROVIDING INFORMATION FOR PREDICTING THERAPEUTIC RESPONSIVENESS TO IMMUNE CHECKPOINT INHIBITOR IN CANCER PATIENT USING MULTIPLE IMMUNOHISTOCHEMICAL STAINING

(71) Applicants: THE ASAN FOUNDATION, Seoul (KR); UNIVERSITY OF ULSAN FOUNDATION FOR INDUSTRY COOPERATION, Ulsan (KR)

(72) Inventors: Sang Yeob KIM, Seoul (KR); Sang We KIM, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/557,802

(22) PCT Filed: Apr. 28, 2022
(86) PCT No.: PCT/KR2022/006075

§ 371 Date: Oct. 27, 2023

(30) Foreign Application Priority Data
Apr. 29, 2021 (KR) 10-2021-0055916

(51) Int. Cl. G01N 33/574; G01N 33/68; G16H 20/10

(52) U.S. Cl. CPC G01N 33/57484; G01N 33/6854; G16H 20/10; G01N 2474/20; G01N 2800/52

(57) ABSTRACT

The present disclosure relates to a method of providing information for predicting a treatment response to an immune checkpoint inhibitor in a cancer patient by using multiplex immunohistochemistry, wherein, by performing multiplex immunohistochemistry on tumor tissue of a cancer patient to measure an expression level of an immune checkpoint molecule by an automated method, the treatment response to the immune checkpoint inhibitor in the cancer patient can be accurately and quickly predicted. In addition, unlike existing methods using single immunohistochemistry, the disclosed method can reduce errors of an inspector by analyzing markers simultaneously expressed in a single cell and evaluating the same by an automated method, and thus will be widely used as a companion diagnostic method for an immune checkpoint inhibitor.

### TECHNICAL FIELD

The present disclosure relates to a method of providing information for predicting a treatment response to an immune checkpoint inhibitor in a cancer patient by using multiplex immunohistochemistry.

### BACKGROUND ART

In the mid-2010s, a critical reason why Keytruda (ingredient name: pembrolizumab) of Merck Company (MSD) was able to beat Opdivo (ingredient name: nivolumab) of BMS Company, which was ahead of development in the immuno-oncology race, was the companion diagnostic criteria.

BMS set a PD-L1 expression threshold at 5% in Phase III clinical trial of testing nivolumab as a first-line treatment for non-small cell lung cancer, but failed to demonstrate superiority over standard chemotherapy in selected patients. Merck, on the other hand, received FDA approval in 2016 for the first-line treatment of non-small cell lung cancer, based on Phase III clinical trial in patients with non-small cell lung cancer in which a PD-L1 threshold was raised to 50%, and significantly increased progression-free survival (PFS) and overall survival (OS) compared to standard chemotherapy. This is an example showing the importance of companion diagnostics.

Meanwhile, companion diagnostics is a biological diagnostic method used to measure a response to a specific drug prescription in a patient, and refers to a diagnostic product developed and released together with pharmaceuticals and to a diagnostic method utilizing the same. Personalized medicine using such companion diagnostics can lead to safer prescriptions that reduce the risk of side effects caused by anticancer drugs.

Currently, many pharmaceutical companies are struggling to develop immuno-oncology therapeutic agents due to the absence of suitable animal models and companion diagnostic evaluation methods. Therefore, there is an urgent need to build a multiplex immunopathology system which enables multi-marker analysis and simultaneous profiling of cells, and a system which enables high-speed imaging of whole slides in large quantities and accurate analysis of the obtained results under the supervision of pathology clinicians. In addition, the demand for building a system that can monitor immunity and make comprehensive determination on a treatment response after prescribing immuno-oncology drugs is rapidly increasing.

Accordingly, the inventors of the present disclosure researched methods of accurately predicting a treatment response to an immune checkpoint inhibitor in a cancer patient, and consequently found that, by performing multiplex immunohistochemistry on cancer tissue to measure an expression level of an immune checkpoint molecule in an automated manner, a response to an immune checkpoint inhibitor could be accurately predicted, thereby completing the present disclosure.

### DISCLOSURE

### Technical Problem

An object of the present disclosure is to provide information for predicting a treatment response to an immune checkpoint inhibitor in a cancer patient.

### Technical Solution

In order to achieve the above object, one aspect provides a method of predicting a treatment response to an immune checkpoint inhibitor in a cancer patient, the method including measuring an expression level of an immune checkpoint molecule by performing multiplex immunohistochemistry on tumor tissue obtained from a cancer patient.

Multiplex immunohistochemistry (multiplex IHC) is a technique capable of simultaneous staining of multiple targets on a single slide. Due to limitations in the use of antibodies in general tissue staining, it is difficult to stain three or more targets on a single slide, but multiplex IHC assays are known to be able to stain up to nine targets simultaneously. In addition, it is difficult to use fluorescent dyes with close spectra in general fluorescence microscopy or confocal microscopy due to bleed-through (a phenomenon in which wavelengths overlap due to excitation cross-talk and emission cross-talk), but the multiplex IHC assays enable imaging with equipment capable of spectrum unmixing using automated algorithms to separate the autofluorescence of tissues as well as the intrinsic fluorescence of each tissue, resulting in accurate imaging. The multiplex IHC assay is a method that acquires information through imaging, automatically segments tissues and cells, automatically analyzes the association between each target and immune cells, and enables large-scale analysis and quantitative analysis of tissue samples.

The term “immune checkpoint inhibitor” refers to a cancer treatment that activates the immune function of immune cells of one's body to fight cancer cells.

The term “treatment response” refers to whether a particular drug, for example, an anticancer drug, exhibits a therapeutic effect on cancer of an individual patient. The term “predicting a treatment response in a cancer patient” may refer to predicting, in advance of administration, whether administration of a therapeutic agent can be useful in the treatment of cancer, and to predicting a treatment response to a therapeutic agent by measuring an expression level of an immune checkpoint molecule. The term “prediction” may refer to pre-determination of a specific outcome, such as a treatment response, through identification of a feature, such as an expression level of an immune checkpoint molecule.

The immune checkpoint molecule may be one or more selected from the group consisting of PD-L1, PD-1 and CTLA-4.

In an embodiment, expression levels of PD-L1 and PD-1 measured from cancer cells and immune cells in tissue from a non-small cell lung cancer patient may be confirmed.

The term “programmed death-ligand 1 (PD-L1)” refers to a protein on the surface of cancer cells or a protein in hematopoietic cells, and is also called CD274 or B7-H1. When proteins on the surface of cancer cells, PD-L1 and PD-L2, bind to a protein on the surface of T cells, PD-1, T cells cannot attack cancer cells. Then, when an immuno-oncology drug binds to a PD-1 receptor on T cells, the evasion function of cancer cells is suppressed. This is how Keytruda and Opdivo work.

The term “programmed death-protein 1 (PD-1)” refers to a protein on the surface of activated T cells (immune cells), and is also called CD279.

The term “cytotoxic T-lymphocyte-associated protein 4 (CTLA-4)” refers to a protein receptor that serves as an immune checkpoint and down-regulates an immune response, and is also called cluster of differentiation 152 (CD152). CTLA4 is expressed intrinsically in regulatory T cells, but is up-regulated only in pre-existing T cells after being activated. This phenomenon is particularly prominent in cancer, and CTLA4 acts as an “off” switch when bound to CD80 or CD86 on the surface of antigen-presenting cells.

The expression level of the immune checkpoint molecule may be measured in cancer cells or immune cells.

The expression level of the immune checkpoint molecules may be measured using a tumor proportion score (TPS) or a combined positive score (TPS).

The term “tumor proportion score (TPS)” refers to a percentage of cancer cells that express immune checkpoint molecules, and the term “combined positive score (CPS)” refers to a percentage of immune cells and cancer cells that express immune checkpoint molecules.

The equation for calculating TPS or CPS is as follows:

TPS=No. PD-L1 positive tumor cells/Total No. of viable tumor cells×100

CPS=No. PD-L1 positive cells(tumor cells, lymphocytes, macrophages)/Total No. of viable tumor cells×100

The multiplex IHC may be performed by using marker antibodies specific for each of cancer cells and immune cells.

In an embodiment, CD4 is a helper T cell, CD8 is a cytotoxic T cell, Foxp3 is a regulatory T cell, pan-cytokeratin (CK) is a tumor cell, DAPI is a nucleus-specific marker, and antibodies against immune checkpoint molecules, PD-1 and PD-L1, may be used to stain tissues of non-small cell lung cancer patients.

Such antibody constitution may be changed to: CD3 (total T cell), CD68 (pan-macrophage), PD-L1, PD-1, CK, and DAPI; or CD8, Foxp3, CD68, PD-1, PD-L1, CK, and DAPI.

The method of predicting a treatment response may include: preprocessing an image in the form of stains obtained by performing multiplex IHC on tumor tissue obtained from a cancer patient; and measuring the expression level of the immune checkpoint molecule by using a machine learning model on the image in the form of staining preprocessed.

The term “machine learning” refers to the improvement of or related study to the information processing ability of computer programs through learning using data and processing experience, and by using a model with a number of parameters, the machine learning may optimize the parameters with the given data.

In the case of companion diagnostics of PD-L1 using the existing single IHC, a pathologist directly determines PD-L1 expression-positive cells and manually measure the number of these cells, and thus there is a problem with variations depending on a pathologist. Therefore, the measuring in an automated manner by machine learning may improve accuracy.

A learning method of the machine learning model may include: a learning method of the machine learning model comprises: generating learning data having as an input condition an image in the form of staining obtained by performing multiplex immunohistochemistry on tumor tissue obtained from a cancer patient and as an output condition an expression level of an immune checkpoint molecule; and iteratively learning a correlation between the image in the form of staining obtained by performing multiplex immunohistochemistry and the expression level of the immune checkpoint molecule, based on the learning data.

In an embodiment, after distinguishing between a tumor cell nest and a stroma and distinguishing between cancer cells and immune cells from the tumor cell nest, the expression level of the immune checkpoint molecule may be measured in each of the cancer cells and immune cells.

The distinguishing between the tumor cell nest and the stroma may be based on a staining type, for example, a region stained with cytokeratin, obtained by performing multiplex IHC.

The distinguishing between the cancer cells and immune cells may be performed by using marker antibodies specific for each of the cancer cells and immune cells.

The staining type may be any one selected from the group consisting of staining intensity, staining location, staining similarity, and autofluorescence.

The staining intensity refers to a degree of staining, and for example, may be divided into weak, mild, strong, and the like. The staining location may be divided into cell membrane, nucleus, cytoplasm, and the like. The staining similarity refers to a degree of similarity compared to a single IHC image. The autofluorescence refers to fluorescence of tissue itself.

The machine learning may include: distinguishing between a tumor cell nest and a stroma; distinguishing between cancer cells and immune cells; and determining whether an immune checkpoint molecule is expressed in each of the cancer cells and immune cells.

The determining of whether the immune checkpoint molecule is expressed may further include determining an expression level of the immune checkpoint molecule by measurement based on TPS or CPS values.

By the method of predicting a treatment response to an immune checkpoint inhibitor in a cancer patient, the expression level of the immune checkpoint molecule may be measured based on TPS or CPS values, and when the TPS or CPS value is at least 15 or more, the method may further include determining that the treatment response to the immune checkpoint inhibitor is high.

In an embodiment, when the TPS or CPS value is at least 15 or more, for example, 16 or more, 17 or more, 18 or more, or 19 or more, it can be determined that the treatment response to the immune checkpoint inhibitor is high.

The cancer may be any one selected from the group consisting of non-small cell lung cancer, small cell lung cancer, melanoma, Hodgkin's lymphoma, stomach cancer, urothelial cell carcinoma, head and neck cancer, liver cancer, colorectal cancer, prostate cancer, pancreatic cancer, liver cancer, testis cancer, ovarian cancer, endometrial cancer, cervical cancer, bladder cancer, brain cancer, breast cancer, and renal cancer, but is not limited thereto.

Redundant content is omitted in consideration of the complexity of the present specification, and terms not otherwise defined in the present specification have meanings commonly used in the technical field to which the present disclosure belongs.

### Advantageous Effects

According to one aspect, multiplex immunohistochemistry is performed on tumor tissue of a cancer patient to measure an expression level of an immune checkpoint molecule by an automated method, so that a treatment response to an immune checkpoint inhibitor in a cancer patient can be accurately and quickly predicted. Also, unlike the existing method using single immunohistochemistry, markers that are simultaneously expressed in a single cell can be analyzed and evaluated so that errors caused by an examiner may be reduced, and thus the method will be widely used as a companion diagnostic method for the immune checkpoint inhibitor.

### DESCRIPTION OF DRAWINGS

FIG. 1 shows a process of performing spectral unmixing for each wavelength band on images obtained by multiplex immunohistochemistry on tumor tissues of a non-small cell lung cancer patient according to an aspect, and FIG. 2 shows the result.

FIG. 3 shows a result of confirming antibody expression for each dye through a pathology view of images obtained by multiplex immunohistochemistry on tumor tissues of a non-small cell lung cancer patient according to an aspect.

FIG. 4 shows a result of performing tissue segmentation through machine learning on images obtained by multiplex immunohistochemistry on tumor tissues of a non-small cell lung cancer patient according to an aspect.

FIG. 5 shows a result of performing cell segmentation through machine learning on images obtained by multiplex immunohistochemistry on tumor tissues of a non-small cell lung cancer patient according to an aspect.

FIG. 6 shows a result of performing phenotyping through machine learning on images obtained by multiplex immunohistochemistry on tumor tissues of a non-small cell lung cancer patient according to an aspect.

FIG. 7 shows a result of quantitative analyzing the results of phenotyping through machine learning on images obtained by multiplex immunohistochemistry on tumor tissues of a non-small cell lung cancer patient according to an aspect.

FIG. 8 shows a result of calculating a TPS value by analyzing results through machine learning on images obtained by multiplex immunohistochemistry on tumor tissues of a non-small cell lung cancer patient according to an aspect.

FIG. 9 shows a result of calculating a CPS value by analyzing results through machine learning on images obtained by multiplex immunohistochemistry on tumor tissues of a non-small cell lung cancer patient according to an aspect.

### BEST MODE

### Mode for Invention

Hereinafter, the present disclosure will be described in more detail with reference to Examples below. However, these Examples are for illustrative purposes only, and the scope of the present disclosure is not intended to be limited by these Examples.

### Example 1. Preparation of Tumor Tissue

Formalin fixation and paraffin embedding (FFPE) of biopsy tissue for diagnosis before drug administration in a non-small cell lung cancer patients were used.

Biopsy tissues from the non-small cell lung cancer patient were fixed by using formalin, and paraffin blocks were fabricated through dehydration, clearing, and paraffin infiltration.

### Example 2. Multiplex Immunohistochemistry

Formalin-fixed, paraffin-embedded blocks of the tumor tissues of the non-small cell lung cancer patient were cut to a thickness of 4 μm, so as to prepare slides. The slides were subjected to multiplex immunofluorescence staining with a Leica Bond Rx™ Automated Stainer (Leica Biosystems, Newcastle, UK).

Specifically, the slides were heated in a drying oven at 60° C. for 30 minutes to melt the paraffin, followed by dewaxing with a Leica Bond Dewax solution (#AR9222, Leica Biosystems), and then, antigen retrieval with Bond Epitope Retrieval 2 (#AR9640, Leica Biosystems) was performed in a pH 9.0 solution for 30 minutes.

After reacting with primary antibodies to a first antigen for 30 minutes, polymer horseradish peroxidase (HRP) Ms+Rb (ARH1001EA, AKOYA Biosciences) was used to react secondary antibodies for 10 minutes. Visualization of the primary antibodies was performed by using fluorescently labeled tyramide signal amplification (TSA, typically diluted 1:150) for 10 minutes, and the slides were then treated with Bond Epitope Retrieval 1 (#AR9961, Leica Biosystems) for 20 minutes to remove bound antibodies before the sequential next step.

For other antigens, visualization was performed in the same manner as in the visualization of the primary antibodies under optimal conditions for each marker. In the case of final antibodies, anti-DIG 780 antibodies were used after labeling with TSA-DIG (diluted at 1:100) for 10 minutes for visualization.

After the multi-marker staining of the slides was finished, the nucleus was finally visualized by staining with DAPI, and the slides were each covered with a cover slip using by using a ProLong Gold anti-fade reagent (P36934, Invitrogen).

### Example 3. Acquisition of Multiplex Immunohistochemistry Images

Slides stained with various antibodies by the method described in Example 2 were scanned by using a Vectra Polaris Automated Quantitative Pathology Imaging System (Akoya Biosciences, Marlborough, MA) to obtain images.

### Example 4. Digital Analysis of Multiplex Immunohistochemistry Images

Images obtained by the method described in Example 3 were analyzed by using the inForm 2.4 software and TIBCO Spotfire™ (Akoya Biosciences, Marlborough, MA) as follows.

A representative slide pf each single spectrum and an unstained tissue slide were used for accurate spectral unmixing. Individually stained slides were each used to establish a fluorescence monospectral library for multispectral analysis. The established spectral library was used to extract fluorescence images corresponding to each marker from the multispectral data by using the spectral unmixing, and each cell was identified by detecting the nuclear spectrum (DAPI).

Tissue segmentation into tumor cell nest and stroma areas was performed by using the inForm image analysis software, and cell segmentation was performed through DAPI staining. Then, an analysis algorithm for each cell was established by using marker staining specific to each immune cell, and phenotyping was performed on a region selected from the scanned slides by using the established algorithm. The phenotyping results were transmitted to the Spotfire™ software to analyze the necessary data.

### Experimental Example 1. Multiplex Immunohistochemistry of Lung Tissues from Non-Small Cell Lung Cancer Patient

Using the method described in Example 2, multiplex immunohistochemistry was performed on each of seven lung tissue slides of a non-small cell lung cancer patient prepared in Example 1 with the configuration described in Table 1 below.

TABLE 1

| | | | |
|---|---|---|---|
| Antibody | Titration | TSA | Titration |

| | |
|---|---|
| Tissue | Lung Pannel 1 |

| | | | | |
|---|---|---|---|---|
| 1^{st} | Foxp3 | 1:100 | Opal480 | 1:300 |
| 2^{nd} | PD-L1 | 1:300 | Opal520 | 1:300 |
| 3^{rd} | CD8 | 1:300 | Opal570 | 1:300 |
| 4^{th} | CD4 | 1:100 | Opal620 | 1:300 |
| 5^{th} | PD-1 | 1:500 | Opal690 | 1:300 |
| 6^{th} | CK | 1:300 | Opal780 | 1:300 |

CD4 is a helper T cell, CD8 is a cytotoxic T cell, Foxp3 is a regulatory T cell, pan-cytokeratin (CK) is a tumor cell, DAPI is a nucleus-specific antibody, and PD-1 and PD-L are immune checkpoint molecules.

### Experimental Example 2. Calculation of TPS and CPS Values Through Digital Analysis of Multiplex Immunohistochemistry Images

The seven slides stained in Experimental Example 1 were scanned by the method described in Example 3 to obtain images, and the data were analyzed by the method described in Example 4.

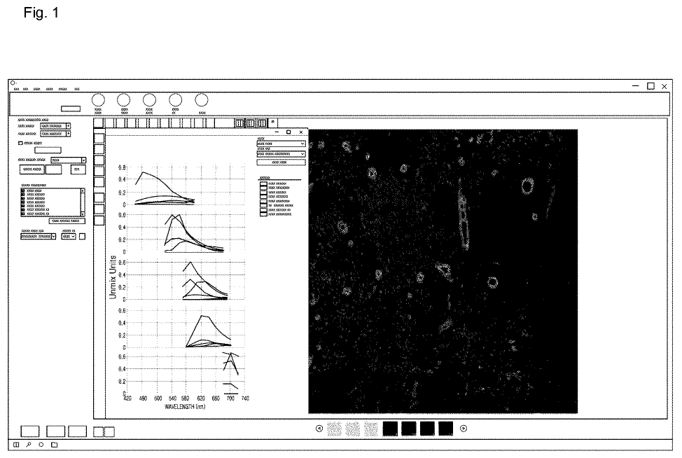

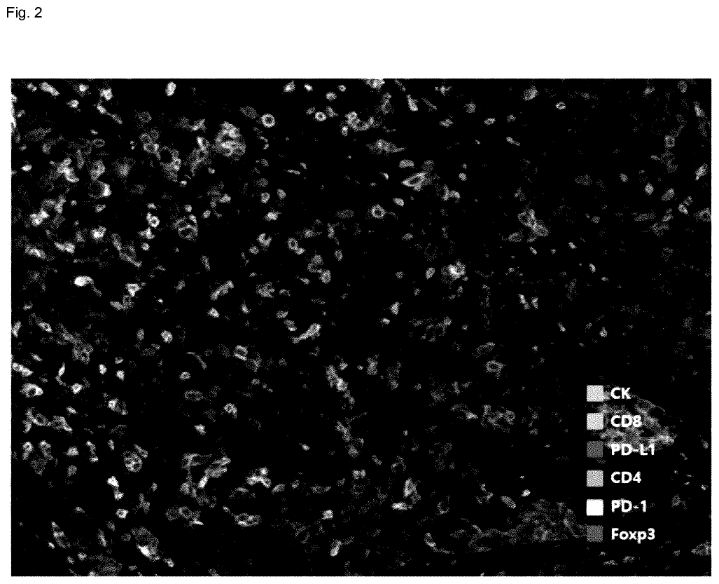

Specifically, as shown in FIG. 1, the slides stained from multiple immunochemistry were subjected to spectral unmixing by the wavelength of each marker (FIG. 2), and the expressions of CK, CD8, PD-L1, CD4, PD-1, and Foxp3 were confirmed through the pathology view (FIG. 3).

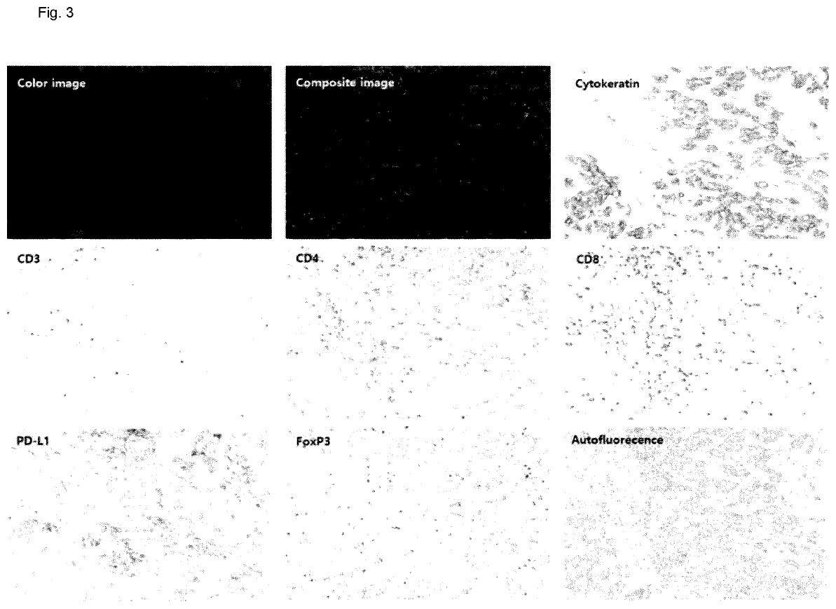

Tissue segmentation was performed through machine learning in which the cytokeratin-stained area was recognized and determined as a tumor cell nest (red) and the other areas were determined as a stroma (green) (FIG. 4).

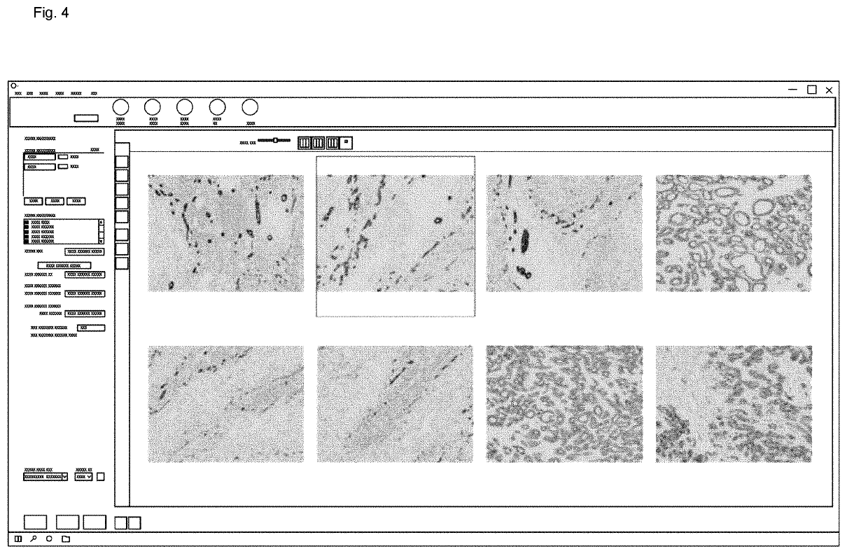

Cell segmentation was performed by using counterstain (DAPI) (FIG. 5). The nucleus shapes of various cells were set to be as accurate as possible to distinguish between cell areas.

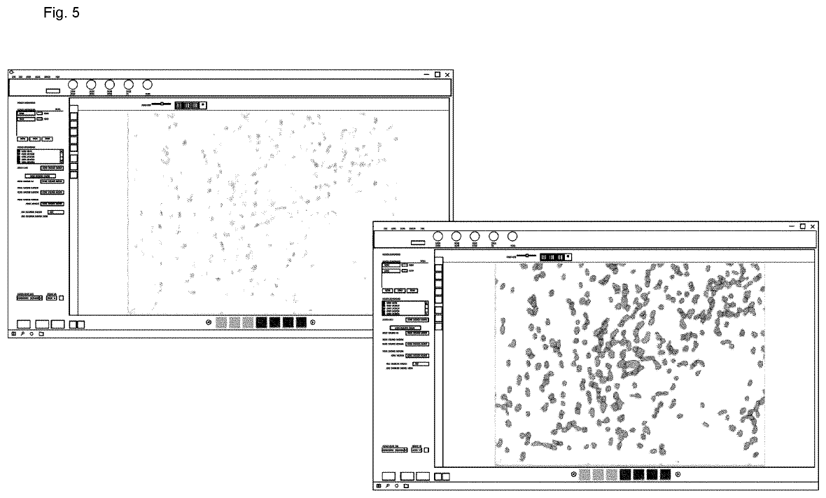

Accordingly, a phenotyping analysis algorithm determining: cytokeratin as positive when stained on the cell membranes mainly in epithelial cells and tumors; CD4 and CD8 as positive when darkly stained on the cell membranes; Foxp3 as positive when darkly stained on the nuclei; and PD-L1 as positive when stained on the cell membranes of immune cells in stroma within intratumoral and peritumoral stroma and the cell membranes of tumor cells. Training was carried out on the entire image area that was not phenotyped through a train classifier using the established analysis algorithm (FIG. 6). The phenotyping was repeated 2 to 3 times, and proceeded until the phenotypes on the analysis image were distinguishable.

Cells determined as positive for cytokeratin were designated as cancer cells, and cells that were co-stained with PD-L1 markers were distinguished from those not co-stained to distinguish between PD-L1-expressing cancer cells and normal cancer cells. Also, cells determined as positive for CD4 were designated as helper T cells, cells determined as positive for CD8 were designated as cytotoxic T cells, and cells determined as positive for Foxp3 were designated as regulatory T cells, and cells that were co-stained with PD-L1 markers were distinguished from those not co-stained to distinguish between PD-L1-expressing immune cells and normal immune cells.

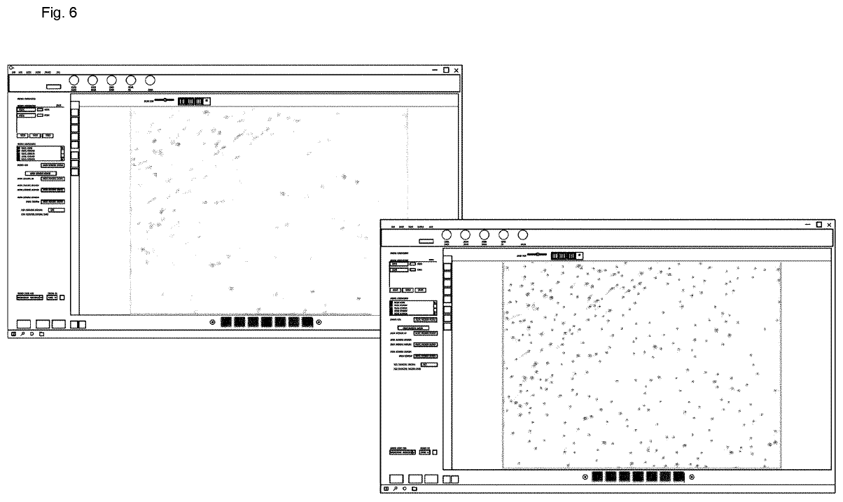

The phenotyped image files were transmitted to the Spotfire™ software to quantify the number of cells that were determined as positive for CD4, CD8, PD-1, Foxp3, and PD-L1 in the tumor cell nest and the stroma (FIG. 7).

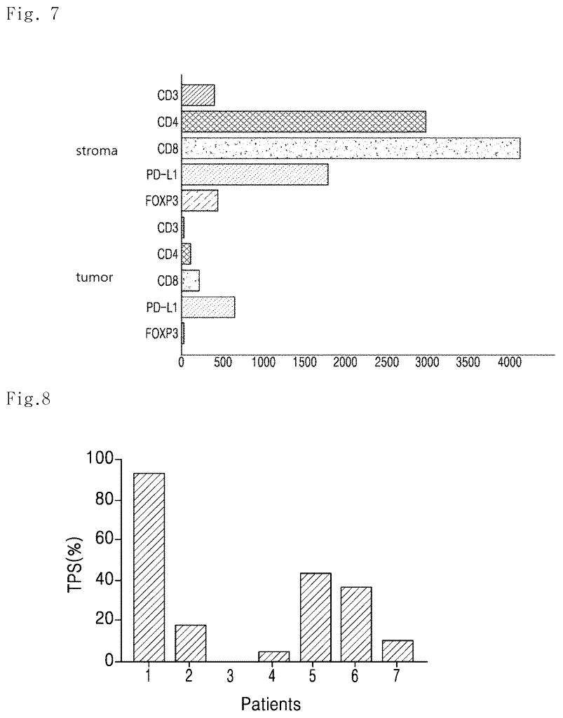

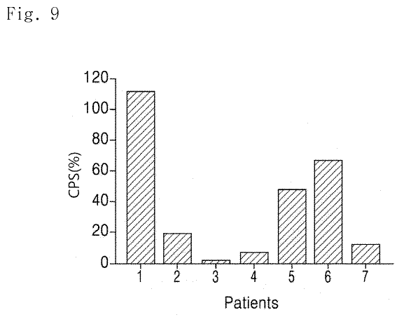

The quantified data values were substituted into equations below to calculate the TPS and CPS values of the seven non-small cell lung cancer patients (FIGS. 8 and 9).

TPS=100*[PD-L1+tumor cell]/[total tumor cell]

CPS=100*([PD-L1+tumor cell]+[PD-L1+helper T cell]+[PD-L1+Treg]+[PD-L1+cytotoxic T cell])/[Total tumor cell]

In addition, the clinical results of the treatment response of the seven non-small cell lung cancer patients administered with immune checkpoint inhibitors, the TPS and CPS values obtained through the above process, and the data compared with the results measured by the existing methods 22C3 and SP23 are presented in Table 2 below.

22C3 (PD-L1 IHC 22C3 pharmDx Overview; Agilent) is a method of diagnosing suitability for administration of Keytruda, which is a PD-1-targeted therapeutic agent, by using single immunohistochemistry for PD-L1, and determines that the patients are suitable for administration of Keytruda when having a cutoff of 50% or more.

VENTANA PD-L1 (SP263) assay (Roche) is used to select patients for administration of IMFINZI or Keytruda through PD-L1 immunohistochemistry of non-small cell lung cancer and urothelial carcinoma tissue, and the administration of Opdivo was used to analyze treatment prognosis. This method should be interpreted by a pathologist, and guides for drug administration are classified for each drug.

TABLE 2

| | | | | | Treatment |
|---|---|---|---|---|---|
| Patient | TPS | CPS | 22C3 | SP263 | response |
| 1 | 93 | 105 | — | 100% | Partial |
| | | | | | remission |
| 2 | 18 | 18 | — | — | Partial |
| | | | | | remission |
| 3 | 1 | 1 | 60% | 50% | Progressive |
| | | | | | lesion |
| 4 | 5 | 5 | 20% | 15% | Progressive |
| | | | | | lesion |
| 5 | 44 | 47 | 80% | 70% | Partial |
| | | | | | remission |
| 6 | 37 | 46 | 60% | 55% | Stable |
| | | | | | lesion |
| 7 | 10 | 11 | 20% | 20% | Progressive |
| | | | | | lesion |

As a result, it was confirmed that patients with high TPS and CPS values measured through the present disclosure mostly showed therapeutic effects in most cases when administered with the immune checkpoint inhibitors. On the other hand, in the case of Patient 3, the analysis result of the present disclosure showed a low value of 1%, but the results of the existing analysis showed a high value of 50% or more, and the treatment response was a progressive lesion, confirming that the prediction of the existing method was not correct. The existing methods are manually performed and rely on the proficiency of pathologists, and in this regard, analyzing the expression levels of PD-L1 in cancer cells and PD-L1 in immune cells with a single marker is confirmed to be less accurate compared to the present disclosure.

1. A method of determining a treatment response to an immune checkpoint inhibitor in a cancer patient, the method comprising:
obtaining a sample of tumor tissue from a cancer patient;
performing multiplex immunohistochemistry on the sample of tumor tissue to obtain an image in the form of staining; and
measuring an expression level of an immune checkpoint molecule from the image.

2. The method of claim 1, wherein the immune checkpoint molecule is at least one selected from the group consisting of PD-L1, PD-1, and CTLA-4.

3. The method of claim 1, wherein the expression level of the immune checkpoint molecule is measured in a cancer cell or an immune cell.

4. The method of claim 1, wherein the expression level of the immune checkpoint molecule is measured using a tumor proportion score (TPS) or a combined positive score (CPS).

5. The method of claim 1, wherein the multiplex immunohistochemistry is performed by staining antibodies specific for each of a cancer cell and an immune cell.

6. The method of claim 1, further comprising:
performing phenotyping through a machine learning model on the image in the form of staining to measure the expression level of the immune checkpoint molecule.

7. The method of claim 6, wherein the form of staining is selected from the group consisting of staining intensity, staining location, staining similarity, and autofluorescence.

8. The method of claim 6, wherein a method of training the machine learning model comprises:
generating learning data having, as an input condition, the image in the form of staining obtained by performing multiplex immunohistochemistry on the tumor tissue obtained from the cancer patient and, as an output condition, the expression level of an immune checkpoint molecule; and
iteratively learning a correlation between the image in the form of staining obtained by performing multiplex immunohistochemistry and the expression level of the immune checkpoint molecule, based on the learning data,
wherein the expression level of the immune checkpoint molecule is measured in each of a cancer cell and an immune cell after distinguishing between a tumor cell nest and a stroma and distinguishing between a cancer cell and an immune cell from the tumor cell nest.

9. The method of claim 1, wherein the cancer patient has a cancer selected from the group consisting of non-small cell lung cancer, small cell lung cancer, melanoma, Hodgkin's lymphoma, stomach cancer, urothelial cell carcinoma, head and neck cancer, liver cancer, colorectal cancer, prostate cancer, pancreatic cancer, testis cancer, ovarian cancer, endometrial cancer, cervical cancer, bladder cancer, brain cancer, breast cancer, and renal cancer.
